(19) United States
(12) Patent Application Publication
(10) Pub. No.: US 2026/0071209 A1
(43) Pub. Date: Mar. 12, 2026

(54) Method for Screening and Rationally Engineering Riboswitch Capable of Specifically Recognizing Doxycycline

(71) Applicant: Jiangnan University, Wuxi (CN)

(72) Inventors: Xiulan SUN, Wuxi (CN); Jiadi SUN, Wuxi (CN); Jingwen ZHOU, Wuxi (CN); Song GAO, Wuxi (CN); Yinzhi ZHANG, Wuxi (CN); Guocheng KOU, Wuxi (CN)

(21) Appl. No.: 19/360,032

(22) Filed: Oct. 16, 2025

(30) Foreign Application Priority Data
Oct. 21, 2024 (CN) 2024114641654

(51) Int. Cl. C12N 15/10; C12N 1/20; C12N 9/10; C12N 15/115; C12N 15/62; C12N 15/70; C12R 1/19

(52) U.S. Cl. CPC C12N 15/1086; C12N 1/20; C12N 9/1055; C12N 15/1058; C12N 15/115; C12N 15/62; C12N 15/70; C07K 2319/60; C12N 2310/16; C12R 2001/19; C12Y 204/0101

(57) ABSTRACT

Disclosed are a method for screening a riboswitch capable of specifically recognizing doxycycline as well as rational engineering and application of the riboswitch, and belongs to the field of gene expression regulation. The present disclosure provides a doxycycline specific riboswitch, and establishes a high-throughput screening method based on flow cytometry, and obtains the doxycycline riboswitch with further improved activation fold by way of computer-aided prediction of binding sites of the riboswitch and calculation of mutation sites. According to a dose-response curve, the riboswitch shows linear correlation at 40-100 μg/L, and meanwhile, a whole-cell sensor constructed is low in price, high in stability and simple to operate, and can be used for on-site rapid detection.

### REFERENCE TO SEQUENCE LISTING

The instant application contains a Sequence-Listing in XML format as a file named “YGHG-2025-25-SEQ.xml” created on Sep. 15, 2025 of 90861 bytes in size, and which is hereby incorporated by reference in its entirety.

### TECHNICAL FIELD

The present disclosure belongs to the field of gene expression regulation, and in particular to a method for screening a riboswitch capable of specifically recognizing doxycycline as well as rational engineering and application of the riboswitch.

### BACKGROUND

Doxycycline is a derivative of tetracycline and a broad-spectrum antibiotic with highly effective antibacterial effects, is widely used to prevent and treat certain bacterial infections, and can also be used as an additive to improve a quality of animal products. However, residues caused by excessive use of doxycycline can accumulate in a body, causing serious impacts on health of humans and animals. With a development of society, requirements for product quality and green environmental protection in a production process are becoming increasingly high, and in order to reduce impacts of residual problems on people's health, detection of doxycycline is receiving more and more attention.

Existing detection methods for doxycycline include high-performance liquid chromatography, high-performance liquid chromatography-tandem mass spectrometry, infrared spectroscopy, gas chromatography-tandem mass spectrometry, and the like. However, these methods have high costs, long detection periods, and low efficiency, and are not suitable for on-site rapid detection of doxycycline. In recent years, sensors have been widely popular due to their high detection efficiency and low cost.

Riboswitches are a new regulatory mode developed in recent years. Riboswitches can specifically sense and recognize small molecule ligands without a need for other cofactors (e.g., proteins, enzymes, metal compounds), and regulate downstream gene expression in processes of transcription and translation. Compared with other regulatory modes such as protein regulation and ribozyme regulation, riboswitches are more sensitive and react faster in recognizing small molecule ligands, and can quickly recognize small molecule ligands under low concentration conditions. Therefore, riboswitches are essentially sensors.

In recent years, riboswitches have been favored by many researchers as an emerging element, but also face many difficulties. Compared with a development of other regulatory elements, a development of riboswitches is still in its infancy, a number of riboswitches developed is limited, and a technology of transforming riboswitches into biosensors is also in its infancy. A traditional artificial riboswitch screening method is a Systematic Evolution of Ligands by Exponential Enrichment (SELEX) technology, which has a history of more than 30 years and has developed relatively maturely. However, screening riboswitches using the technology requires in vitro enrichment screening for high affinity aptamers, followed by in vivo high-throughput screening to select sequences capable of adapting to an intracellular environment. An entire process is relatively time-consuming and labor-intensive.

Therefore, it is urgent to screen out doxycycline riboswitches to be engineered into biosensors capable of monitoring doxycycline residues in food and other environments in real-time.

### SUMMARY

In response to the aforementioned shortcomings of the prior art, the present disclosure provides a riboswitch capable of specifically recognizing doxycycline and a method for screening and rationally engineering the riboswitch. The riboswitch capable of specifically recognizing doxycycline is screened by flow cytometry, and the riboswitch can specifically recognize doxycycline.

A first technical solution provided by the present disclosure is a method for screening a riboswitch capable of specifically recognizing doxycycline, including the following steps:
(1) constructing a doxycycline riboswitch library through an RNA aptamer for doxycycline;
(2) constructing a riboswitch reporting platform by fusing SacB gene with EGFP gene;
(3) constructing a plasmid library using the doxycycline riboswitch library from step (1) and the riboswitch reporting platform from step (2);
(4) transforming the plasmid library from step (3) into Escherichia coli to be co-incubated with a substrate; and collecting high-fluorescent cells, i.e., positive cells, by flow cytometry;
(5) cultivating the positive cells from step (4) by spreading on a plate, and picking white nonfluorescent single colonies, i.e., positive colonies; and
(6) cultivating the positive colonies from step (5) on a well plate, measuring a fluorescence intensity at an excitation wavelength of 488 nm and an emission wavelength of 520 nm, and measuring OD₆₀₀ using a spectrophotometer, calculating a fluorescence value per unit cell, and screening out a cell with a higher fluorescence value per unit, i.e., the cell containing the riboswitch capable of specifically recognizing doxycycline.

In some embodiments, in step (1), a nucleotide sequence of the RNA aptamer for doxycycline is as follows:

(SEQ ID NO. 2)
GGGAGACGCGAAAGCGUUACGAAUGCGAUGACUCGUCGAAA
GACGAACAGUUCCUUUGGAUCCGAAUUCGCCGC.

In some embodiments, the doxycycline riboswitch library is constructed by inserting 10 random bases N into a 3′ end of a DNA sequence of doxycycline through the RNA aptamer for doxycycline, and the doxycycline riboswitch library is shown as SEQ ID NO. 3:

(SEQ ID NO. 3)
GGGAGACGCGAAAGCGTTACGAATGCGATGACTCGTCGAAAG
ACGAACAGTTCCTTTGGATCCGAATTCGCCGCNNNNNNNNNN.

In some embodiments, in step (2), SacB-21-EGFP fusion protein, i.e., the riboswitch reporting platform, is constructed by fusing a 21 bp fragment of the SacB gene, the nucleotide sequence of which is shown as SEQ ID NO. 4, with the EGFP gene.

In some embodiments, in step (3), the plasmid library is constructed by inserting the doxycycline riboswitch library between a promoter and a ribosome binding site of a recombinant plasmid containing the riboswitch reporting platform.

In some embodiments, the promoter is a T7 strong promoter, and the ribosome binding site is AAGGAG.

In some embodiments, the recombinant plasmid uses PET-Duet-1 as an expression vector.

In some embodiments, in step (4), the Escherichia coli includes E. coli JM109 and E. coli BL21.

In some embodiments, in step (4), an IPTG concentration for inducing expression of the Escherichia coli is 0.5 mM.

In some embodiments, in step (4), the substrate is doxycycline with a concentration of 100 μg/L.

In some embodiments, a fermentation culture is always maintained at a temperature of 37° C. and a rotational speed of 220 rpm.

In some embodiments, a culture medium is LB medium.

In some embodiments, a pH of the culture medium is 6-7.

A second technical solution provided by the present disclosure is a riboswitch (DOX-3-4) capable of specifically recognizing doxycycline, and a nucleotide sequence of the riboswitch is shown as SEQ ID NO. 1:

DOX-3-4 (SEQ ID NO. 1):
GGGAGACGCGAAAGCGUUACGAAUGCGAUGACUCGUCGAAAGA
CGAACAGUUCCUUUGGAUCCGAAUUCGCCGC-CACGAUUUGU.

A third technical solution provided by the present disclosure is a biosensor carrying the riboswitch described in the second technical solution.

A fourth technical solution provided by the present disclosure is a method for engineering a riboswitch for doxycycline, and the method includes mutating a 26^{th} nucleotide, a 47^{th} nucleotide, a 51^{st} nucleotide, and/or a 53^{rd} nucleotide of a parent of the riboswitch, a nucleotide sequence of the parent being shown as SEQ ID NO. 1.

In some embodiments, a mutation is obtained by mutating the parent as shown in any one of (1) to (6):
(1) mutating a 26^{th} cytosine C to guanine G;
(2) mutating a 47^{th} adenine A to uracil U;
(3) mutating a 49^{th} adenine A to guanine G;
(4) mutating a 51^{st} uracil U to cytosine C;
(5) mutating a 53^{th} cytosine C to guanine G;
(6) mutating the 26^{th} cytosine C to guanine G, and mutating the 47^{th} adenine A to uracil U.

A fifth technical solution provided by the present disclosure is an application of the aforementioned riboswitch, the aforementioned biosensor, or the aforementioned method in synthesis of doxycycline.

The present disclosure has the following technical effects:
1. The present disclosure is a method for screening a doxycycline riboswitch based on flow cytometry. Through extensive literature review, an aptamer for doxycycline was found, and only 10 random bases were ligated following the aptamer, thereby reducing a library capacity and improving a success rate of screening.
2. A host for screening the doxycycline riboswitch in the present disclosure is E. coli, and downstream expressed genes are controlled by the strong T7 promoter and a strong AAGGAG ribosome binding site, and an appropriate IPTG concentration is selected to greatly reduce a probability of false positives.
3. A entire process of the present disclosure is based on a screening process in vivo, and the screened riboswitches have high adaptability in vivo.
4. The riboswitches screened out by the present disclosure are subjected to dual verification of a dose-response curve and molecular docking to investigate the interaction between the riboswitches and ligands.
5. The riboswitches screened out by the present disclosure have a high sensing ability for doxycycline, but have no recognition effect on other small molecule ligands.
6. The mutant riboswitches constructed by the present disclosure can further increase an activation fold of the riboswitches.

### BRIEF DESCRIPTION OF FIGURES

FIG. 1 is a diagram showing plasmid construction for a riboswitch library;

FIG. 2 shows verification of tolerance to a doxycycline substrate;

FIG. 3 shows optimization of an IPTG concentration;

FIG. 4 shows is a diagram showing results of fusion of SacB conserved with EGFP;

FIG. 5 shows a positive control image of flow cytometry;

FIG. 6 shows results of collection by flow cytometry;

FIG. 7 is a diagram showing results of high-throughput screening;

FIG. 8 shows a dose-response curve and activation folds of a riboswitch;

FIG. 9 shows prediction of a secondary structure and a tertiary structure of a riboswitch;

FIG. 10 shows direct molecular docking between a riboswitch and doxycycline;

FIG. 11 shows results of single point mutation of a riboswitch;

FIG. 12 shows results of combinatorial mutation of a riboswitch;

FIG. 13 shows a dose-response curve of a mutant riboswitch; and

FIG. 14 shows molecular docking of a mutant riboswitch.

### DETAILED DESCRIPTION

The preferred examples of the present disclosure will be described below. It should be understood that the examples are for better explaining the present disclosure and are not intended to limit the present disclosure.

### Testing Methods:

Measurement of fluorescence intensity: 200 μl of a fermented bacterial solution was taken into a black 96-well plate. A fluorescence intensity of cells was measured under an excitation wavelength of 488 nm and an emission wavelength of 520 nm.

Measurement of OD₆₀₀: 20 μl of a fermented bacterial solution was taken into a white 96-well plate, and the bacterial solution was diluted 10-fold with 180 μl of deionized water and an absorbance was measured at 600 nm.

Raw materials used in the examples:
1. PET-Duet-1 vector, from our laboratory.
2. Escherichia coli BL21 (DE3), from our laboratory.
3. Formula for LB liquid culture medium: peptone (10 g), yeast powder (5 g), and sodium chloride (10 g), 1 L in volume.
4. Formula for LB solid culture medium: peptone (10 g), yeast powder (5 g), and sodium chloride (10 g), 1 L in volume, aliquoted into 5 shake flasks, with 3.5 g of agar powder added.

### Example 1: Establishment of High-Throughput Screening Solution

### (I) Establishment of High-Throughput Screening Solution

### (1) Expression of Fluorescent Reporter Gene EGFP

EGFP fluorescent protein gene (SEQ ID NO. 5) stored in the laboratory was ligated to a PET-Duet-1 vector by homologous recombination using primers in Table 1. A plasmid was extracted and sequenced to obtain a recombinant plasmid PET-Duet-EGFP. The recombinant plasmid PET-Duet-EGFP was transformed into E. coli BL21 (DE3) to obtain a recombinant strain BL21/PET-Duet-EGFP.

TABLE 1
Primers and EGFP fluorescent protein genes

| Name of primers | Sequence 5′-3′ |
|---|---|
| PET-F | atacaaataatctactagcgcagcttaatt |
| (SEQ ID NO. 11) | aacctaggctgctgcc |
| PET-R | ccttacccatggagatgctccttcctatag |
| (SEQ ID NO. 12) | tgagtcgtattaatttcg |
| EGFP-F | gagcatctccatgggtaagggagaagaact |
| (SEQ ID NO. 13) | tttcactggagttgtcc |
| EGFP-R | cgctagtagattatttgtatagttcatcca |
| (SEQ ID NO. 14) | tgccatgtgtaatcccagatgggtaaggga |
| | gaagaacttttcactggagttgtcccaatt |
| | cttgttgaattagatggtgatgttaatggg |
| | cacaaattttctgtcagtggagagggtgaa |
| | ggtgatgcaacatacggaaaacttaccctt |
| | aaatttatttgcactactggaaagcttcct |
| | gttccttggccaacacttgtcactactctt |
| | acttatggtgttcaatgcttttcaagatac |
| | ccagatcatatgaagcggcacgacttcttc |
| | aagagcgccatgcctgagggatacgtgcag |
| | ga |
| EGFP | gaggaccatcttcttcaaggacgacgggaa |
| (SEQ ID NO. 5) | ctacaagacacgtgctgaagtcaagtttga |
| | gggagacaccctcgtcaacagaatcgagct |
| | taagggaatcgatttcaaggaggacggaaa |
| | catcctcggccacaagttggaatacaacta |
| | caactcccacaacgtatacatcatggcaga |
| | caaacaaaagaatggaatcaaagttaactt |
| | caaaattagacacaacattgaagatggaag |
| | cgttcaactagcagaccattatcaacaaaa |
| | tactccaattggcgatggccctgtcctttt |
| | accagacaaccattacctgtccacacaatc |
| | tgccctttcgaaagatcccaacgaaaagag |
| | agaccacatggtccttcttgagtttgtaac |
| | agctgctgggattacacatggcatggatga |
| | actatacaaataa |

### (2) Verification of Tolerance to a Doxycycline Substrate

A doxycycline solution was prepared at a concentration of 50 mg/L, and different volumes of pre-prepared substrates were added when the doxycycline solution was poured into a plate to form different concentration gradients. The recombinant strain BL21/PET-Duet-EGFP was cultivated for 12 h, then diluted 10,000-fold, aliquoted in a volume of 30 μL, and evenly spread on pre-prepared plates. After 12 h of cultivation, the cells were observed for the growth condition to determine the concentration for screening.

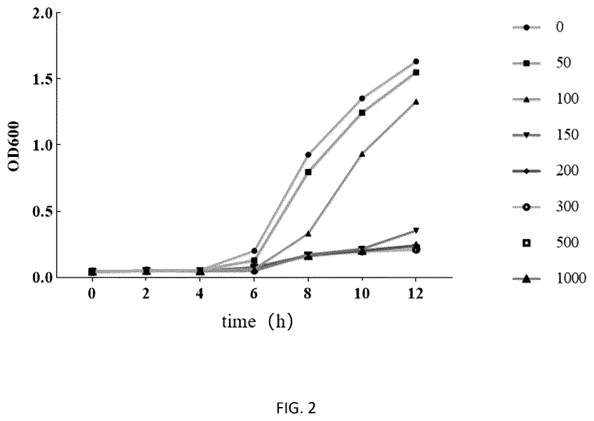

As shown in FIG. 2, in the verification of the tolerance to the doxycycline substrate, when the concentration was greater than 100 μg/L, the cells grew slowly or did not grow. This is because doxycycline could bind to ribosomes, and thus inhibited protein synthesis and prevented bacterial growth. Therefore, the concentration of 100 μg/L was selected as the concentration for subsequent high-throughput screening.

### (3) Optimization of IPTG Concentration

The IPTG concentration was optimized by a well plate fermentation method. The recombinant plasmid PET-Duet-EGFP was transformed into E. coli BL21 (DE3) to obtain the recombinant strain BL21/PET-Duet-EGFP. 0 mM, 0.25 mM, 0.5 mM, 0.75 mM, 1 mM, 1.5 mM, 1.75, and 2 mM IPTG was added to the culture medium and cultivated at 37° C. and 220 rpm for 12 h. Fluorescence values were measured using a multifunctional microplate reader at an excitation wavelength of 488 nm and an emission wavelength of 520 nm. At the same time, the OD₆₀₀ of the cells was measured. According to the fluorescence value per unit cell=the fluorescence value/the OD₆₀₀, the fluorescence values per unit cell of the experimental group and the control group were calculated, and the IPTG concentration was determined.

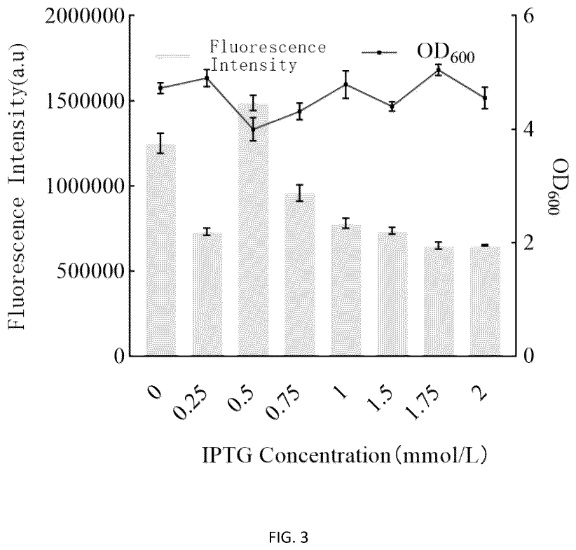

As shown in FIG. 3, during optimization of the IPTG concentration, it was found that under the induction of IPTG of 0.5 mM, the fluorescence value per unit cell was the highest. Therefore, 0.5 mM was selected as the concentration for subsequent induction.

### (4) Optimization of Fusion Sequence

To achieve complete expression of the fluorescent reporter gene, attempts were made to conserve some of the bases of the SacB gene (SEQ ID NO. 7) for ligation, and the fluorescence values were measured. The numbers of bases of the SacB gene conserved were set to 9, 21, 30, 42, and 60 (SEQ ID NOS. 6, 4, 8, 9, and 10), respectively. Using primers in Table 2, genes were ligated to PET-Duet-EGFP plasmids by homologous recombination, to construct 5 plasmids PET-Duet-SacB-9-EGFP, PET-Duet-SacB-21-EGFP, PET-Duet-SacB-30-EGFP, PET-Duet-SacB-42-EGFP, and PET-Duet-SacB-60-EGFP. The aforementioned plasmids were transformed into E. coli BL21 (DE3) to obtain recombinant strain BL21/PET-Duet-SacB-9-EGFP, recombinant strain BL21/PET-Duet-SacB-21-EGFP, recombinant strain BL21/PET-Duet-SacB-30-EGFP, recombinant strain BL21/PET-Duet-SacB-42-EGFP, and recombinant strain BL21/PET-Duet-SacB-60-EGFP. Then, the aforementioned recombinant strains were fermented at 37° C. and 220 rpm for 12 h. Following cultivation, fluorescence values were measured using a microplate reader at an excitation wavelength of 488 nm and an emission wavelength of 520 nm. At the same time, the OD₆₀₀ of the cells was measured. According to the fluorescence value per unit cell=the fluorescence value/the OD₆₀₀, the fluorescence values per unit cell of the experimental group and the control group were calculated.

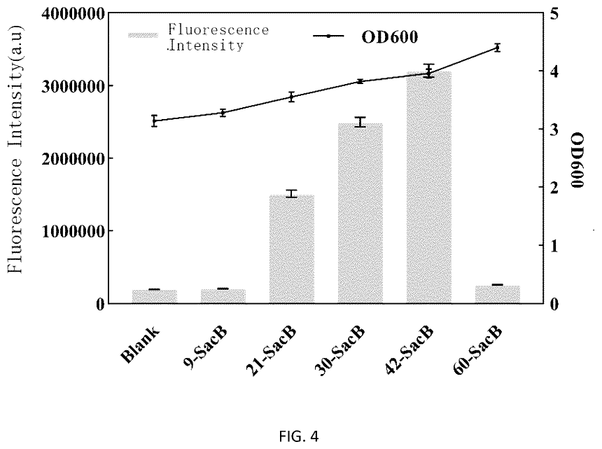

As shown in FIG. 4 which is a diagram showing results of fusion of SacB conserved with EGFP, when the number of bases fused was 21, the fluorescence value per unit cell increased significantly, and the fusion mode (i.e., the plasmid PET-Duet-SacB-21-EGFP) could be selected for high-throughput screening.

TABLE 2
Genes and primers

| Gene/fragment | Sequence |
|---|---|
| SacB-9 | Gcgagtgaaggagcatctcc-atgaacatc- |
| (SEQ ID NO. 6) | ggtaagggagaagaactttt |
| SacB-9-F | ggtaagggagaagaacttttcactggagttg |
| (SEQ ID NO. 15) | tcccaattc |
| SacB-9-R | ggagatgctccttcactcgcatttggtcatg |
| (SEQ ID NO. 16) | tgatcggc |
| SacB-21 | Gcgagtgaaggagcatctcc-atgaacatca |
| (SEQ ID NO. 4) | aaaagtttgca-ggtaagggagaagaactttt |
| SacB-21-F | ggtaagggagaagaacttttcactggagttgt |
| (SEQ ID NO. 17) | cccaattc |
| SacB-21-R | ggagatgctccttcactcgcatttggtcatgt |
| (SEQ ID NO. 18) | gatcggc |
| SacB-30 | Gcgagtgaaggagcatctcc-atgaacatcaa |
| (SEQ ID NO. 8) | aaagtttgcaaaacaagca-ggtaagggagaa |
| | gaactttt |
| SacB-30-F | ggtaagggagaagaacttttcactggagttgt |
| (SEQ ID NO. 19) | cccaattc |
| SacB-30-R | ggagatgctccttcactcgcatttggtcatgt |
| (SEQ ID NO. 20) | gatcggc |
| SacB-42 | Gcgagtgaaggagcatctcc-atgaacatcaa |
| (SEQ ID NO. 9) | aaagtttgcaaaacaagcaacagtattaacc- |
| | ggtaagggagaagaactttt |
| SacB-42-F | ggtaagggagaagaacttttcactggagttg |
| (SEQ ID NO. 21) | tcccaattc |
| SacB-42-R | ggagatgctccttcactcgcatttggtcatg |
| (SEQ ID NO. 22) | tgatcggc |
| SacB-60 | Gcgagtgaaggagcatctcc-atgaacatca |
| (SEQ ID NO. 10) | aaaagtttgcaaaacaagcaacagtattaac |
| | ctttactaccgcactgctg-ggtaagggaga |
| | agaactttt |
| SacB-60-F | ggtaagggagaagaacttttcactggagttg |
| (SEQ ID NO. 23) | tcccaattc |
| SacB-60-R | ggagatgctccttcactcgcatttggtcatg |
| (SEQ ID NO. 24) | tgatcggc |
| SacB | atgaacatcaaaaagtttgcaaaacaagcaa |
| (SEQ ID NO. 7) | cagtattaacctttactaccgcactgctgg |
| | caggaggcgcaactcaagcgtttgcgaaag |
| | aaacgaaccaaaagccatataaggaaacat |
| | acggcatttcccatattacacgccatgata |
| | tgctgcaaatccctgaacagcaaaaaaatg |
| | aaaaatatcaagttcctgaattcgattcgt |
| | ccacaattaaaaatatctcttctgcaaaag |
| | gcctggacgtttgggacagctggccattac |
| | aaaacgctgacggcactgtcgcaaactatc |
| | acggctaccacatcgtctttgcattagccg |
| | gagatcctaaaaatgcggatgacacatcga |
| | tttacatgttctatcaaaaagtcggcgaaa |
| | cttctattgacagctggaaaaacgctggcc |
| | gcgtctttaaagacagcgacaaattcgatg |
| | caaatgattctatcctaaaagaccaaacac |
| | aagaatggtcaggttcagccacatttacat |
| | ctgacggaaaaatccgtttattctacactg |
| | atttctccggtaaacattacggcaaacaaa |
| | cactgacaactgcacaagttaacgtatcag |
| | catcagacagctctttgaacatcaacggtg |
| | tagaggattataaatcaatctttgacggtg |
| | acggaaaaacgtatcaaaatgtacagcagt |
| | tcatcgatgaaggcaactacagctcaggcg |
| | acaaccatacgctgagagatcctcactacg |
| | tagaagataaaggccacaaatacttagtat |
| | ttgaagcaaacactggaactgaagatggct |
| | accaaggcgaagaatctttatttaacaaag |
| | catactatggcaaaagcacatcattcttcc |
| | gtcaagaaagtcaaaaacttctgcaaagcg |
| | ataaaaaacgcacggctgagttagcaaacg |
| | gcgctctcggtatgattgagctaaacgatg |
| | attacacactgaaaaaagtgatgaaaccgc |
| | tgattgcatctaacacagtaacagatgaaa |
| | ttgaacgcgcgaacgtctttaaaatgaacg |
| | gcaaatggtacctgttcactgactcccgcg |
| | gatcaaaaatgacgattgacggcattacgt |
| | ctaacgatatttacatgcttggttatgttt |
| | ctaattctttaactggcccatacaagccgc |
| | tgaacaaaactggccttgtgttaaaaatgg |
| | atcttgatcctaacgatgtaacctttactt |
| | actcacacttcgctgtacctcaagcgaaag |
| | gaaacaatgtcgtgattacaagctatatga |
| | caaacagaggattctacgcagacaaacaat |
| | caacgtttgcgccaagcttcctgctgaaca |
| | tcaaaggcaagaaaacatctgttgtcaaag |
| | acagcatccttgaacaaggacaattaacag |
| | ttaacaaataa |

### (5) Design of Positive Control by Flow Cytometry

1 ml of a solution of the recombinant strain BL21/PET-Duet-SacB-21-EGFP was taken and added to 4 mL of LB (containing ampicillin and 0.5 mM IPTG) liquid culture medium, and cultivated at 37° C. and 220 rpm for 16 h. The cultivated bacterial solution was washed twice with PBS buffer and diluted until the OD was 0.3. The fluorescence intensity was measured by flow cytometry.

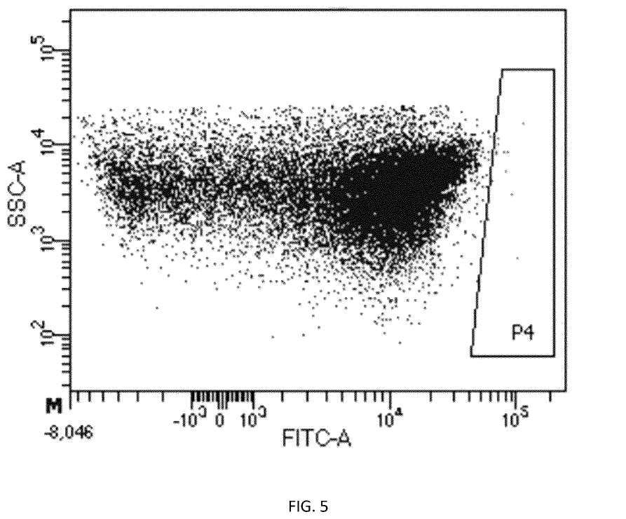

As shown in FIG. 5 which shows a positive control image of flow cytometry, as a subsequent positive reference, only cells with fluorescence values greater than that of the positive control were collected when sample cells were collected.

### (II) Construction of Riboswitch Library

(1) Design of riboswitch plasmid library

Random ssDNA library and primers (synthesized by Shanghai Sangon Bioengineering Co., Ltd.):

5′-GGGAGACGCGAAAGCGTTACGAATGCGATGACTCGTCGAAAGACGAACAGTTCCTTTGGATCCGAATT CGCCGC-N10-aaggagcatctccatgaaca-3′ (SEQ ID NO. 25), where N10 represents a sequence composed of 10 random nucleotide bases ligated together, with the underlined indicating the ribosome binding site.

Forward primer:
(SEQ ID NO. 26)
5′-aaggagcatctccatgaacatcaaaaagtttgcaggtaag-3′;
and
Reverse primer:
(SEQ ID NO. 27)
5′-GCGGCGAATTCGGATCCAAAGGAACTGTTCGTCTTTCGAC-3′.

Both the random ssDNA library and the primers were prepared into stock solutions of 100 μM using BB buffer (Tris-HCl: 20 Mm, MgCl₂: 50 mM, KCl: 5 mM, and CaCl₂): 2 Mm, pH 7.6), and the stock solutions were stored at −20° C. for future use.

### (2) Construction of Riboswitch Plasmid Library by PCR Amplification

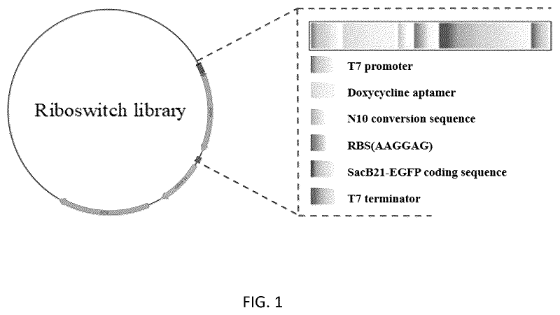

The riboswitch plasmid library
GGGAGACGCGAAAGCGTTACGAATGCGATGACTCGTCGAAAGACGAACAGTTCCTTTG GATCCGAATTCGCCGC-N10 (SEQ ID NO. 3) was inserted between the promoter and the ribosome binding site of the recombinant plasmid PET-Duet-SacB-21-EGFP by homologous recombination. The riboswitch plasmid library was constructed by PCR amplification using the forward and reverse primers in (1) and referring to Tables 3 and 4, as shown in FIG. 1.

TABLE 3
PCR system

| Component | Volume | |
|---|---|---|
| 2xPhanta Max Master Mix | 25 | μL |
| Forward primer | 2 μL (100 μM) | |
| Reverse primer | 2 μL (100 μM) | |
| Ultrapure water | 20 | μL |
| Template DNA | 1 | μL |

TABLE 4
PCR procedure

| Step | Conditions |
|---|---|
| 1 | 95° C., 5 min |
| 2 | 95° C., 15 s |
| | 57° C., 30 s |
| | 72° C., 4 min |
| 3 | 72° C., 5 min |
| | 4° C., ∞ |

Verification by polyacrylamide gel electrophoresis: the PCR product was subjected to electrophoresis using 2% agarose gel.

The plasmid library was transformed into E. coli BL21 (DE3) to construct recombinant E. coli containing the plasmid library.

### Example 2: High-Throughput Screening of Doxycycline Riboswitch

1 ml of a solution of the recombinant E. coli containing the plasmid library in Example 1 was taken and added to 4 mL of LB (containing ampicillin, 0.5 mM IPTG, and 100 μg/L doxycycline) liquid culture medium, and cultivated at 37° C. and 220 rpm for 16 h. The cultivated bacterial solution was washed twice with PBS buffer and diluted until the OD was 0.3. High-fluorescent cells were collected from the finally obtained cells by flow cytometry. The collected high-fluorescent cells were spread onto LB (containing ampicillin and 0.5 mM IPTG) solid culture medium, and cultivated at 37° C. for 12 h.

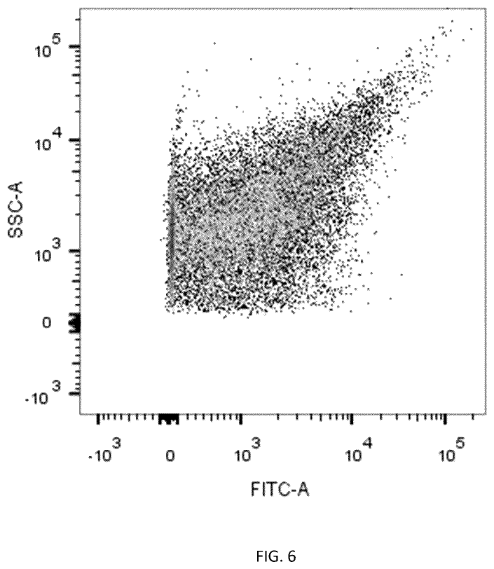

As shown in FIG. 6 which shows results of collection by flow cytometry, only cells with fluorescence values greater than 3*104 were collected.

Then, under UV irradiation, white cells were picked and cultivated in a new LB (containing ampicillin) solid culture medium at 37° C. for 12 h. The cultivated cells were seeded into 48-well plates (control group: containing ampicillin and 0.5 mM IPTG; experimental group: containing ampicillin, 0.5 mM IPTG, and 100 μg/L doxycycline), and cultivated at 37° C. and 220 rpm for 12 h. Following cultivation, fluorescence values were measured using a microplate reader at an excitation wavelength of 488 nm and an emission wavelength of 520 nm. At the same time, the OD₆₀₀ of the cells was measured. According to the fluorescence value per unit cell=the fluorescence value/the OD₆₀₀, the fluorescence values per unit cell of the experimental group and the control group were calculated.

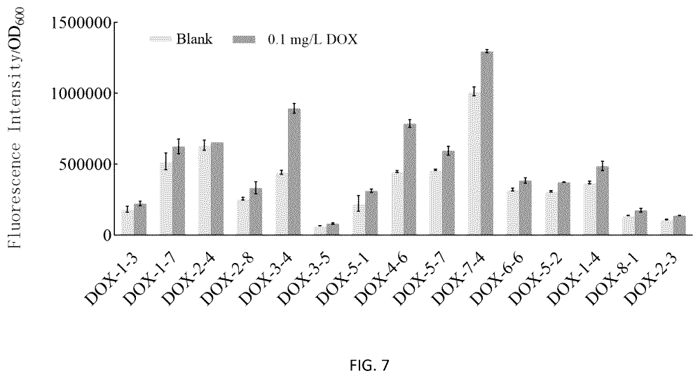

As shown in FIG. 7 which shows results of high-throughput screening, it was found that the DOX-3-4 riboswitch had a significant difference in the fluorescence values per unit cell between the experimental group and the control group, and could be used as a candidate riboswitch.

DOX-3-4 cells were picked and cultivated in a new LB (containing ampicillin) solid culture medium at 37° C. for 12 h. The cultivated cells were seeded into 48-well plates (control group: containing ampicillin and 0.5 mM IPTG; experimental group: containing ampicillin, 0.5 mM IPTG, and 100 μg/L doxycycline), and three parallel groups were set up for the experimental group and the control group, respectively. Following cultivation, fluorescence values were measured at an excitation wavelength of 488 nm and an emission wavelength of 520 nm. At the same time, the OD₆₀₀ of the cells was measured. DOX-3-4 cells were cultivated in a new LB (containing ampicillin) liquid culture medium at 37° C. and 220 rpm for 12 h. Following cultivation, the plasmid DOX-3-4-PET-Duet-SacB-21-EGFP was extracted and sent for sequencing. The correct sequence was the riboswitch sequence SEQ ID NO. 1

(GGGAGACGCGAAAGCGUUACGAAUGCGAUGACUCGUCGAAAGA
CGAACAGUUCCUUUGGAUCCGAAUUCGCCGC-CACGAUUUGU).

### Example 3: Analysis of Dose-Response Curves of Riboswitches

The dose-response curves of riboswitches were determined by a well plate fermentation method, with concentrations set as 0 μg/L, 25 μg/L, 50 μg/L, and 100 μg/L to measure the dose-response curve. DOX-3-4 cells were cultivated on 48-well plates respectively, where the control group contained ampicillin and 0.5 mM IPTG, and the experimental group contained ampicillin, 0.5 mM IPTG, and different concentrations of doxycycline. Following cultivation, the fluorescence intensity was measured at an excitation wavelength of 488 nm and an emission wavelength of 520 nm, and the OD₆₀₀ was measured using a spectrophotometer to calculate the fluorescence value per unit cell. The dose-response curves were created using GraphPad Prism 8.0 software.

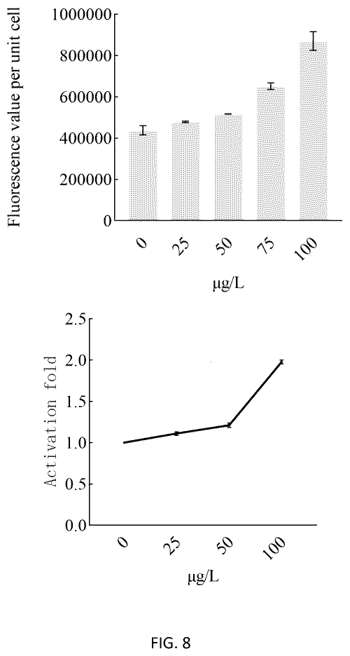

As shown in FIG. 8, the dose-response curves and the activation folds of the riboswitches were determined respectively. Under the action of 100 μg/L doxycycline, the riboswitches were activated 1.99-fold.

### Example 4: Computer Aided Design of Mutation Sites

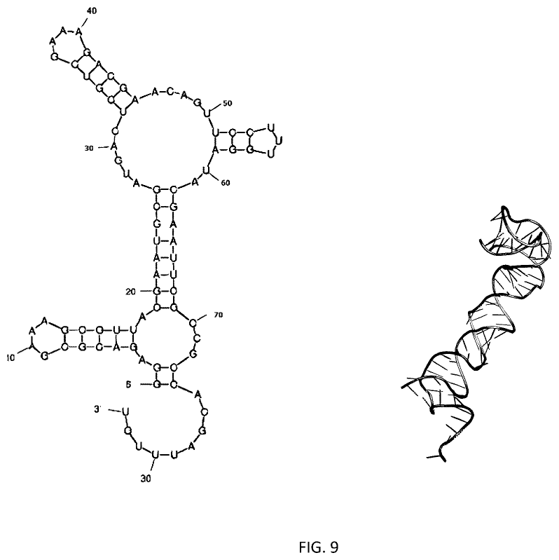

A secondary structure of riboswitch RNA was predicted using Mfold (RNA Folding Form (unafold.org)) online software. The principle of prediction of the software is the principle of minimizing free energy, and the software has been continuously developed over decades and is relatively mature. A tertiary structure of riboswitch RNA was predicted using the 3d RNA (Xiao Lab (hust.edu.cn)) online software developed by Huazhong University of Science and Technology. The software can accurately predict the tertiary spatial structure of RNA or DNA in a short time. Prediction of the secondary structure and the tertiary structure of the riboswitch is shown in FIG. 9.

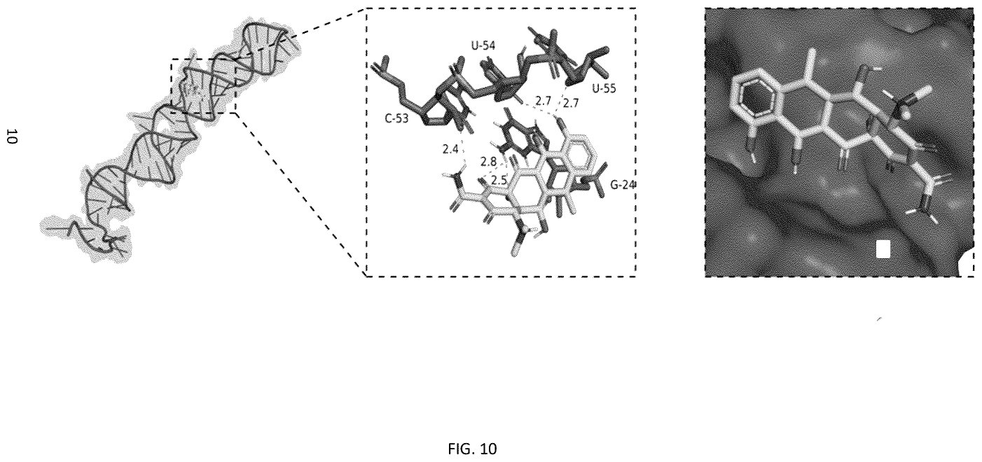

Large molecule riboswitches and small molecule ligands were subjected to molecular docking using Autodock4. Before docking using the software, PDB files for both large molecules and small molecules were to be prepared, where the large molecules could be directly predicted and saved in PDB format using online software, while the secondary structure of the small molecules could be saved in SDF format and finally converted to PDB format using Open Bable software. The complete steps of the software were as follows: large molecules and small molecules were prepared; the large molecules and the small molecules were preprocessed; a docking box was set up; Autogrid was run; and Autodock was run. Generally, the results of docking for 50 times were relatively accurate. Following docking, the docking results were viewed in an Analze box, and the one with the lowest binding free energy was selected, saved, and opened in pymol software to visualize an image and display a binding pocket. Direct molecular docking between the riboswitch and doxycycline is shown in FIG. 10.

### Example 5: Engineering of Riboswitch Performance by Single Point Mutation Combined with Combinatorial Mutation

Hotspot bases within a binding site region were identified, and single point saturated mutation (including any nucleotide at positions 22-30, nucleotide at position 41, nucleotide at position 42, or any nucleotide at positions 45-55) was conducted. Iterative combinatorial mutation was conducted on mutants that are beneficial for single point saturated mutation, and whether the mutants are beneficial was determined by the activation fold.

TABLE 5
Design of primers for single point mutation

| Primer | 5′-3′ |
|---|---|
| 22-F | GGAGACGCGAAAGCGTTACGANTGCGATGA |
| (SEQ ID NO. 28) | CTCGTCGAAAG |
| 23-F | GGAGACGCGAAAGCGTTACGAANGCGATGA |
| (SEQ ID NO. 29) | CTCGTCGAAAG |
| 24-F | GGAGACGCGAAAGCGTTACGAATNCGATGA |
| (SEQ ID NO. 30) | CTCGTCGAAAG |
| 25-F(SEQ ID NO. 31) | GGAGACGCGAAAGCGTTACGAATGNGATGA |
| | CTCGTCGAAAG |
| 26-F | GGAGACGCGAAAGCGTTACGAATGCNATGA |
| (SEQ ID NO. 32) | CTCGTCGAAAG |
| 27-F | GGAGACGCGAAAGCGTTACGAATGCGNTGA |
| (SEQ ID NO. 33) | CTCGTCGAAAG |
| 28-F | GGAGACGCGAAAGCGTTACGAATGCGANGA |
| (SEQ ID NO. 34) | CTCGTCGAAAG |
| 29-F | GGAGACGCGAAAGCGTTACGAATGCGATNA |
| (SEQ ID NO. 35) | CTCGTCGAAAG |
| 30-F | GGAGACGCGAAAGCGTTACGAATGCGATGN |
| (SEQ ID NO. 36) | CTCGTCGAAAG |
| 22-30-R | CGTAACGCTTTCGCGTCTCCCcctatagtg |
| (SEQ ID NO. 37) | agtcg |
| 41-F | ACGAATGCGATGACTCGTCGAAANACGAAC |
| (SEQ ID NO. 38) | AGTTCC |
| 42-F | ACGAATGCGATGACTCGTCGAAAGNCGAAC |
| (SEQ ID NO. 39) | AGTTCC |
| 41-42-R | CGACGAGTCATCGCATTCGTAACGCTTTCG |
| (SEQ ID NO. 40) | CGTCTCC |
| 45-F | CGATGACTCGTCGAAAGACGNACAGTTCCT |
| (SEQ ID NO. 41) | TTGGATCCG |
| 46-F | CGATGACTCGTCGAAAGACGANCAGTTCCT |
| (SEQ ID NO. 42) | TTGGATCCG |
| 47-F | CGATGACTCGTCGAAAGACGAANAGTTCCT |
| (SEQ ID NO. 43) | TTGGATCCG |
| 48-F | CGATGACTCGTCGAAAGACGAACNGTTCCT |
| (SEQ ID NO. 44) | TTGGATCCG |
| 49-F | CGATGACTCGTCGAAAGACGAACANTTCCT |
| (SEQ ID NO. 45) | TTGGATCCG |
| 50-F | CGATGACTCGTCGAAAGACGAACAGNTCCT |
| (SEQ ID NO. 46) | TTGGATCCG |
| 51-F | CGATGACTCGTCGAAAGACGAACAGTNCCT |
| (SEQ ID NO. 47) | TTGGATCCG |
| 52-F | CGATGACTCGTCGAAAGACGAACAGTTNCT |
| (SEQ ID NO. 48) | TTGGATCCG |
| 53-F | CGATGACTCGTCGAAAGACGAACAGTTCNT |
| (SEQ ID NO. 49) | TTGGATCCG |
| 54-F | CGATGACTCGTCGAAAGACGAACAGTTCCN |
| (SEQ ID NO. 50) | TTGGATCCG |
| 55-F | CGATGACTCGTCGAAAGACGAACAGTTCCT |
| (SEQ ID NO. 51) | NTGGATCCG |
| 45-55-R | CGTCTTTCGACGAGTCATCGCATTCGTAAC |
| (SEQ ID NO. 52) | GCTTTCGC |

TABLE 6
Design of primers for combinatorial mutation

| Primer | 5′-3′ |
|---|---|
| AB-F | GATGACTCGTCGAAAGACGATCAGTTCCTT |
| (SEQ ID NO. 53) | TGGATCCG |
| AB-R | TCGTCTTTCGACGAGTCATCCCATTCGTAA |
| (SEQ ID NO. 54) | CGCTTTCG |
| AC-F | GATGACTCGTCGAAAGACGAACGGTTCCTT |
| (SEQ ID NO. 55) | TGGATCCG |
| AC-R | TCGTCTTTCGACGAGTCATCCCATTCGTAA |
| (SEQ ID NO. 56) | CGCTTTCGCG |
| AD-F | TGACTCGTCGAAAGACGAACAGCTCCTTTG |
| (SEQ ID NO. 57) | GATCCGAATTC |
| AD-R | GTTCGTCTTTCGACGAGTCATCCCATTCGT |
| (SEQ ID NO. 58) | AACGCTTTCG |
| AE-F | CTCGTCGAAAGACGAACAGTTGCTTTGGAT |
| (SEQ ID NO. 59) | CCGAATTCG |
| AE-R | ACTGTTCGTCTTTCGACGAGTCATCCCATT |
| (SEQ ID NO. 60) | CGTAACGC |
| BC-F | GATGACTCGTCGAAAGACGATCGGTTCCTT |
| (SEQ ID NO. 61) | TGGATCCG |
| AA-R | TCGTCTTTCGACGAGTCATCGCATTCGTAA |
| (SEQ ID NO. 62) | CGCTTTCGC |
| BD-F | GATGACTCGTCGAAAGACGATCAGCTCCTT |
| (SEQ ID NO. 63) | TGGATCCG |
| BE-F | GATGACTCGTCGAAAGACGATCAGTTGCTT |
| (SEQ ID NO. 64) | TGGATCCG |
| CD-F | GATGACTCGTCGAAAGACGAACGGCTCCTT |
| (SEQ ID NO. 65) | TGGATCCG |
| CE-F | GATGACTCGTCGAAAGACGAACGGTTGCTT |
| (SEQ ID NO. 66) | TGGATCCG |
| DE-F | GATGACTCGTCGAAAGACGAACAGCTGCTT |
| (SEQ ID NO. 67) | TGGATCCG |
| ABC-F | TGACTCGTCGAAAGACGATCGGTTCCTTTG |
| (SEQ ID NO. 68) | GATCCG |
| ABC-R | GATCGTCTTTCGACGAGTCATCCCATTCGT |
| (SEQ ID NO. 69) | AACGCTTTC |
| ABD-F | ATGACTCGTCGAAAGACGATCAGCTCCTTT |
| (SEQ ID NO. 70) | GGATCCG |
| ABD-R | ATCGTCTTTCGACGAGTCATCCCATTCGTA |
| (SEQ ID NO. 71) | ACGCTTTCG |
| ABE-F | ATGACTCGTCGAAAGACGATCAGTTGCTTT |
| (SEQ ID NO. 72) | GGATCCG |
| ABE-R | ATCGTCTITCGACGAGTCATCCCATTCGTA |
| (SEQ ID NO. 73) | ACGCTTTCGCG |
| ACD-F | GTCGAAAGACGAACGGCTCCTTTGGATCCG |
| (SEQ ID NO. 74) | AATTCGCC |
| ACD-R | GGAGCCGTTCGTCTITCGACGAGTCATCCC |
| (SEQ ID NO. 75) | ATTCGTAAC |
| ACE-F | CGAAAGACGAACGGTTGCTTTGGATCCGAA |
| (SEQ ID NO. 76) | TTCGCCGC |
| ACE-R | AAGCAACCGTTCGTCTTTCGACGAGTCATC |
| (SEQ ID NO. 77) | CCATTCGTAAC |
| ADE-F | GACTCGTCGAAAGACGAACAGCTGCTTTGG |
| (SEQ ID NO. 78) | ATCCGAATTC |
| ADE-R | TGTTCGTCTTTCGACGAGTCATCCCATTCG |
| (SEQ ID NO. 79) | TAACGCTTTC |
| BCD-F | CTCGTCGAAAGACGATCGGCTCCTTTGGAT |
| (SEQ ID NO. 80) | CCGAATTC |
| BCD-R | GCCGATCGTCTTTCGACGAGTCATCGCATT |
| (SEQ ID NO. 81) | CGTAACG |
| BCE-F | ATGACTCGTCGAAAGACGATCGGTTGCTTT |
| (SEQ ID NO. 82) | GGATCCG |
| BCE-R | ATCGTCTTTCGACGAGTCATCGCATTCGTA |
| (SEQ ID NO. 83) | ACGCTTTC |
| CDE-F | CGTCGAAAGACGAACGGCTGCTTTGGATCC |
| (SEQ ID NO. 84) | GAATTCGCCG |
| CDE-R | CAGCCGTTCGTCTTTCGACGAGTCATCGCA |
| (SEQ ID NO. 85) | TTCGTAACGC |
| ABCD-F | TGACTCGTCGAAAGACGATCGGCTCCTTTG |
| (SEQ ID NO. 86) | GATCCG |
| ABCD-R | GATCGTCTTTCGACGAGTCATCCCATTCGT |
| (SEQ ID NO. 87) | AACGCTTTCG |
| ABCE-F | CTCGTCGAAAGACGATCGGTTGCTTTGGAT |
| (SEQ ID NO. 88) | CCGAATTCGC |
| ABCE-R | ACCGATCGTCTTTCGACGAGTCATCCCATT |
| (SEQ ID NO. 89) | CGTAACGC |
| ABDE-F | TGGGATGACTCGTCGAAAGACGATCAGCTG |
| (SEQ ID NO. 90) | CTTTGGATC |
| ABDE-R | TCTTTCGACGAGTCATCCCATTCGTAACGC |
| (SEQ ID NO. 91) | TTTCGCG |
| BCDE-F | TCGTCGAAAGACGATCGGCTGCTTTGGATC |
| (SEQ ID NO.92) | CGAATTCGC |
| BCDE-R | AGCCGATCGTCTTTCGACGAGTCATCGCAT |
| (SEQ ID NO. 93) | TCGTAACG |
| ACDE-F | TGACTCGTCGAAAGACGAACGGCTGCTTTG |
| (SEQ ID NO. 94) | GATCCGAATTCG |
| ACDE-R | GTTCGTCTTTCGACGAGTCATCCCATTCGT |
| (SEQ ID NO. 95) | AACGCTTTC |
| ABCDE-F | GACTCGTCGAAAGACGATCGGCTGCTTTGG |
| (SEQ ID NO. 96) | ATCCGAATTC |
| ABCDE-R | CGATCGTCTTTCGACGAGTCATCCCATTCG |
| (SEQ ID NO. 97) | TAACGCTTTC |

The specific methods are as follows:

Using full plasmid PCR technology, the DOX-3-4-PET-Duet-SacB-21-EGFP plasmid was subjected to site directed mutation using the aforementioned primers to obtain a mutant containing plasmid (mutant DOX-3-4-PET-Duet-SacB-21-EGFP). The aforementioned plasmid was transformed into E. coli BL21 (D3), and the activation fold of the riboswitch was determined.

The aforementioned recombinant strain was seeded into 48-well plates (control group: containing ampicillin and 0.5 mM IPTG; experimental group: containing ampicillin, 0.5 mM IPTG, and 100 μg/L doxycycline), and cultivated at 37° C. and 220 rpm for 12 h. Following cultivation, fluorescence values were measured using a microplate reader at an excitation wavelength of 488 nm and an emission wavelength of 520 nm. At the same time, the OD₆₀₀ of the cells was measured. According to the fluorescence value per unit cell=the fluorescence value/the OD₆₀₀, the fluorescence values per unit cell of the experimental group and the control group were calculated.

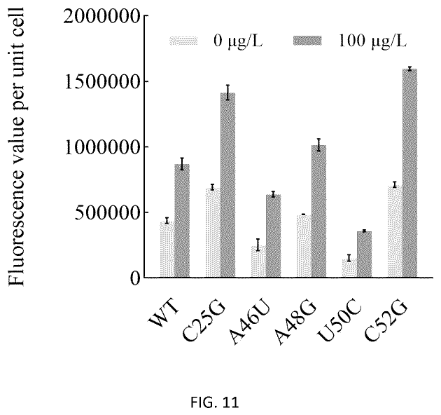

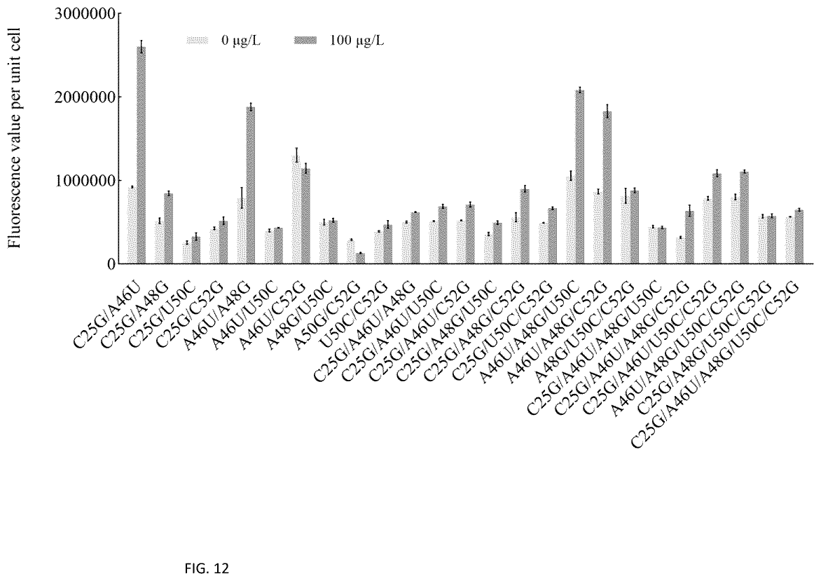

As shown in FIG. 11 and FIG. 12, which show results of single point mutation and combinatorial mutation of riboswitches, single point mutation was performed first, and five mutant riboswitches with increased single point mutation effects were found, namely C26G, A47U, A49G, U51C, and C53G. Then, combinatorial mutation was performed on the five mutants, and it was found that the C26G/A47U mutant had a higher activation fold when combined.

### Example 6: Analysis of Dose-Response Curve Based on Mutant Riboswitch

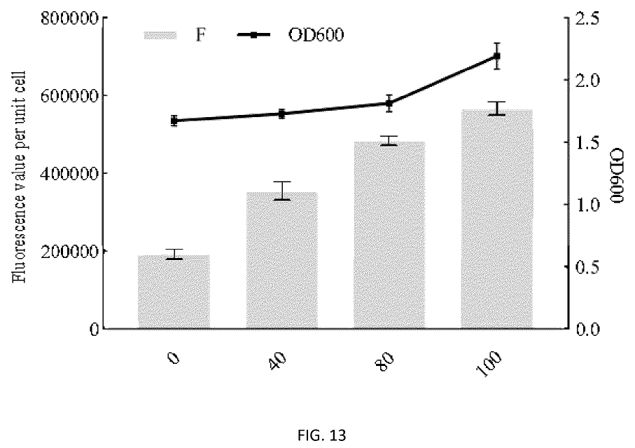

First, the dose-response curve of the C26G/A47U combinatorial mutant riboswitch obtained in Example 5 was determined by a well plate fermentation method. The control group was set to contain ampicillin and 0.5 mM IPTG, and the experimental group was set to contain ampicillin, 0.5 mM IPTG, and different concentrations of doxycycline. Then, the dose-response curve of the C26G/A47U mutant riboswitch was determined by a shake flask fermentation method. As shown in FIG. 13, the dose-response curve of the mutant riboswitch showed an increase in activation fold of the riboswitch from 1.99 to 2.78.

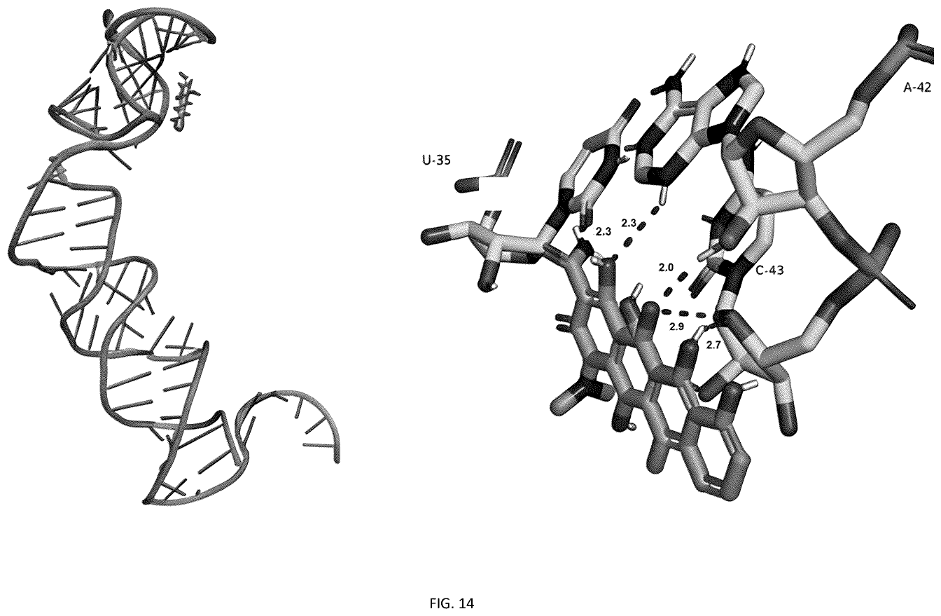

Molecular docking was conducted referring to the method of Example 4, the results shown in FIG. 14, and it was found that the binding energy changed from −3.11 kcal/mol to −6.48 kcal/mol, forming a more stable complex system.

Although the present disclosure has been disclosed as above in exemplary examples, it is not intended to limit the present disclosure. Anyone familiar with this technology can make various changes and modifications without departing from the spirit and scope of the present disclosure. Therefore, the protection scope of the present disclosure shall be as defined in the claims.

What is claimed is:

1. A method for screening a riboswitch capable of specifically recognizing doxycycline, comprising the following steps:
(1) constructing a doxycycline riboswitch library through an RNA aptamer for doxycycline;
(2) constructing a riboswitch reporting platform by fusing SacB gene with EGFP gene;
(3) constructing a plasmid library using the doxycycline riboswitch library from step (1) and the riboswitch reporting platform from step (2);
(4) transforming the plasmid library from step (3) into Escherichia coli to be co-incubated with a substrate, and collecting high-fluorescent cells which are positive cells by flow cytometry;
(5) cultivating the positive cells from step (4) by spreading on a plate, and picking white nonfluorescent single colonies which are positive colonies; and
(6) cultivating the positive colonies from step (5) on a well plate, measuring a fluorescence intensity at an excitation wavelength of 488 nm and an emission wavelength of 520 nm, and measuring OD₆₀₀ using a spectrophotometer, calculating a fluorescence value per unit cell, and screening out a cell with a higher fluorescence value per unit, which is the cell carrying the riboswitch capable of specifically recognizing doxycycline.

2. The method according to claim 1, wherein in step (1), a nucleotide sequence of the RNA aptamer for doxycycline is set forth in SEQ ID NO:2.

3. The method according to claim 1, wherein in step (1), the doxycycline riboswitch library is constructed by inserting 10 random bases N into a 3′ end of a DNA sequence of doxycycline through the RNA aptamer for doxycycline, and the doxycycline riboswitch library is set forth in SEQ ID NO:3.

4. The method according to claim 1, wherein in step (2), SacB-21-EGFP fusion protein which is the riboswitch reporting platform, is constructed by fusing a 21 bp fragment of the SacB gene with the EGFP gene, a nucleotide sequence of the 21 bp fragment of the SacB gene is set forth in SEQ ID NO:4, and a nucleotide sequence of the EGFP gene is set forth in SEQ ID NO:5.

5. The method according to claim 1, wherein in step (3), the plasmid library is constructed by inserting the doxycycline riboswitch library between a promoter and a ribosome binding site of a recombinant plasmid containing the riboswitch reporting platform.

6. The method according to claim 5, wherein the promoter is a T7 strong promoter, the ribosome binding site is AAGGAG, and the recombinant plasmid uses PET-Duet-1 as an expression vector.

7. The method according to claim 5, wherein in step (4), the Escherichia coli comprises E. coli JM109 and E. coli BL21.

8. The method according to claim 5, wherein in step (4), an IPTG concentration for inducing expression of the Escherichia coli is 0.5 mM.

9. The method according to claim 5, wherein in step (4), the substrate is doxycycline with a concentration of 100 μg/L.

10. A riboswitch capable of specifically recognizing doxycycline screened out by the method according to claim 1, wherein a nucleotide sequence of the riboswitch is set forth in SEQ ID NO: 1.

11. A method for engineering a riboswitch for doxycycline, wherein the method comprises mutating a 26^{th} nucleotide, a 47^{th} nucleotide, a 51^{st} nucleotide, and/or a 53^{rd} nucleotide of a parent of the riboswitch according to claim 10.

12. The method according to claim 11, wherein the parent is mutated as shown in any one of (1) to (6):
(1) mutating a 26^{th} cytosine C to guanine G;
(2) mutating a 47^{th} adenine A to uracil U;
(3) mutating a 49^{th} adenine A to guanine G;
(4) mutating a 51^{st} uracil U to cytosine C;
(5) mutating a 53^{th} cytosine C to guanine G;
(6) mutating the 26^{th} cytosine C to guanine G, and mutating the 47^{th} adenine A to uracil U.


SEQUENCE LISTING (97 sequences)

SEQ ID NO: 1 - RNA, 84 nt, synthetic construct
  source 1..84: mol_type = other RNA
gggagacgcgaaagcgttacgaatgcgatgactcgtcgaaagacgaacagttcctttggatccgaattcgccgccacgatttgt

SEQ ID NO: 2 - RNA, 74 nt, synthetic construct
  source 1..74: mol_type = other RNA
gggagacgcgaaagcgttacgaatgcgatgactcgtcgaaagacgaacagttcctttggatccgaattcgccgc

SEQ ID NO: 3 - DNA, 84 bp, synthetic construct
  source 1..84: mol_type = other DNA
gggagacgcgaaagcgttacgaatgcgatgactcgtcgaaagacgaacagttcctttggatccgaattcgccgcnnnnnnnnnn

SEQ ID NO: 4 - DNA, 61 bp, synthetic construct
  source 1..61: mol_type = other DNA
gcgagtgaaggagcatctccatgaacatcaaaaagtttgcaggtaagggagaagaactttt

SEQ ID NO: 5 - DNA, 717 bp, synthetic construct
  source 1..717: mol_type = other DNA
atgggtaagggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaagcttcctgttccttggccaacacttgtcactactcttacttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacagaatcgagcttaagggaatcgatttcaaggaggacggaaacatcctcggccacaagttggaatacaactacaactcccacaacgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 6 - DNA, 49 bp, synthetic construct
  source 1..49: mol_type = other DNA
gcgagtgaaggagcatctccatgaacatcggtaagggagaagaactttt

SEQ ID NO: 7 - DNA, 1422 bp, synthetic construct
  source 1..1422: mol_type = other DNA
atgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataa

SEQ ID NO: 8 - DNA, 70 bp, synthetic construct
  source 1..70: mol_type = other DNA
gcgagtgaaggagcatctccatgaacatcaaaaagtttgcaaaacaagcaggtaagggagaagaactttt

SEQ ID NO: 9 - DNA, 82 bp, synthetic construct
  source 1..82: mol_type = other DNA
gcgagtgaaggagcatctccatgaacatcaaaaagtttgcaaaacaagcaacagtattaaccggtaagggagaagaactttt

SEQ ID NO: 10 - DNA, 100 bp, synthetic construct
  source 1..100: mol_type = other DNA
gcgagtgaaggagcatctccatgaacatcaaaaagtttgcaaaacaagcaacagtattaacctttactaccgcactgctgggtaagggagaagaactttt

SEQ ID NO: 11 - DNA, 46 bp, synthetic construct
  source 1..46: mol_type = other DNA
atacaaataatctactagcgcagcttaattaacctaggctgctgcc

SEQ ID NO: 12 - DNA, 48 bp, synthetic construct
  source 1..48: mol_type = other DNA
ccttacccatggagatgctccttcctatagtgagtcgtattaatttcg

SEQ ID NO: 13 - DNA, 47 bp, synthetic construct
  source 1..47: mol_type = other DNA
gagcatctccatgggtaagggagaagaacttttcactggagttgtcc

SEQ ID NO: 14 - DNA, 48 bp, synthetic construct
  source 1..48: mol_type = other DNA
cgctagtagattatttgtatagttcatccatgccatgtgtaatcccag

SEQ ID NO: 15 - DNA, 40 bp, synthetic construct
  source 1..40: mol_type = other DNA
ggtaagggagaagaacttttcactggagttgtcccaattc

SEQ ID NO: 16 - DNA, 39 bp, synthetic construct
  source 1..39: mol_type = other DNA
ggagatgctccttcactcgcatttggtcatgtgatcggc

SEQ ID NO: 17 - DNA, 40 bp, synthetic construct; identical to SEQ ID NO: 15
  source 1..40: mol_type = other DNA

SEQ ID NO: 18 - DNA, 39 bp, synthetic construct; identical to SEQ ID NO: 16
  source 1..39: mol_type = other DNA

SEQ ID NO: 19 - DNA, 40 bp, synthetic construct; identical to SEQ ID NO: 15
  source 1..40: mol_type = other DNA

SEQ ID NO: 20 - DNA, 39 bp, synthetic construct; identical to SEQ ID NO: 16
  source 1..39: mol_type = other DNA

SEQ ID NO: 21 - DNA, 40 bp, synthetic construct; identical to SEQ ID NO: 15
  source 1..40: mol_type = other DNA

SEQ ID NO: 22 - DNA, 39 bp, synthetic construct; identical to SEQ ID NO: 16
  source 1..39: mol_type = other DNA

SEQ ID NO: 23 - DNA, 40 bp, synthetic construct; identical to SEQ ID NO: 15
  source 1..40: mol_type = other DNA

SEQ ID NO: 24 - DNA, 39 bp, synthetic construct; identical to SEQ ID NO: 16
  source 1..39: mol_type = other DNA

SEQ ID NO: 25 - DNA, 104 bp, synthetic construct
  source 1..104: mol_type = other DNA
gggagacgcgaaagcgttacgaatgcgatgactcgtcgaaagacgaacagttcctttggatccgaattcgccgcnnnnnnnnnnaaggagcatctccatgaaca

SEQ ID NO: 26 - DNA, 40 bp, synthetic construct
  source 1..40: mol_type = other DNA
aaggagcatctccatgaacatcaaaaagtttgcaggtaag

SEQ ID NO: 27 - DNA, 40 bp, synthetic construct
  source 1..40: mol_type = other DNA
gcggcgaattcggatccaaaggaactgttcgtctttcgac

SEQ ID NO: 28 - DNA, 41 bp, synthetic construct
  source 1..41: mol_type = other DNA
ggagacgcgaaagcgttacgantgcgatgactcgtcgaaag

SEQ ID NO: 29 - DNA, 41 bp, synthetic construct
  source 1..41: mol_type = other DNA
ggagacgcgaaagcgttacgaangcgatgactcgtcgaaag

SEQ ID NO: 30 - DNA, 41 bp, synthetic construct
  source 1..41: mol_type = other DNA
ggagacgcgaaagcgttacgaatncgatgactcgtcgaaag

SEQ ID NO: 31 - DNA, 41 bp, synthetic construct
  source 1..41: mol_type = other DNA
ggagacgcgaaagcgttacgaatgngatgactcgtcgaaag

SEQ ID NO: 32 - DNA, 41 bp, synthetic construct
  source 1..41: mol_type = other DNA
ggagacgcgaaagcgttacgaatgcnatgactcgtcgaaag

SEQ ID NO: 33 - DNA, 41 bp, synthetic construct
  source 1..41: mol_type = other DNA
ggagacgcgaaagcgttacgaatgcgntgactcgtcgaaag

SEQ ID NO: 34 - DNA, 41 bp, synthetic construct
  source 1..41: mol_type = other DNA
ggagacgcgaaagcgttacgaatgcgangactcgtcgaaag

SEQ ID NO: 35 - DNA, 41 bp, synthetic construct
  source 1..41: mol_type = other DNA
ggagacgcgaaagcgttacgaatgcgatnactcgtcgaaag

SEQ ID NO: 36 - DNA, 41 bp, synthetic construct
  source 1..41: mol_type = other DNA
ggagacgcgaaagcgttacgaatgcgatgnctcgtcgaaag

SEQ ID NO: 37 - DNA, 35 bp, synthetic construct
  source 1..35: mol_type = other DNA
cgtaacgctttcgcgtctccccctatagtgagtcg

SEQ ID NO: 38 - DNA, 36 bp, synthetic construct
  source 1..36: mol_type = other DNA
acgaatgcgatgactcgtcgaaanacgaacagttcc

SEQ ID NO: 39 - DNA, 36 bp, synthetic construct
  source 1..36: mol_type = other DNA
acgaatgcgatgactcgtcgaaagncgaacagttcc

SEQ ID NO: 40 - DNA, 37 bp, synthetic construct
  source 1..37: mol_type = other DNA
cgacgagtcatcgcattcgtaacgctttcgcgtctcc

SEQ ID NO: 41 - DNA, 39 bp, synthetic construct
  source 1..39: mol_type = other DNA
cgatgactcgtcgaaagacgnacagttcctttggatccg

SEQ ID NO: 42 - DNA, 39 bp, synthetic construct
  source 1..39: mol_type = other DNA
cgatgactcgtcgaaagacgancagttcctttggatccg

SEQ ID NO: 43 - DNA, 39 bp, synthetic construct
  source 1..39: mol_type = other DNA
cgatgactcgtcgaaagacgaanagttcctttggatccg

SEQ ID NO: 44 - DNA, 39 bp, synthetic construct
  source 1..39: mol_type = other DNA
cgatgactcgtcgaaagacgaacngttcctttggatccg

SEQ ID NO: 45 - DNA, 39 bp, synthetic construct
  source 1..39: mol_type = other DNA
cgatgactcgtcgaaagacgaacanttcctttggatccg

SEQ ID NO: 46 - DNA, 39 bp, synthetic construct
  source 1..39: mol_type = other DNA
cgatgactcgtcgaaagacgaacagntcctttggatccg

SEQ ID NO: 47 - DNA, 39 bp, synthetic construct
  source 1..39: mol_type = other DNA
cgatgactcgtcgaaagacgaacagtncctttggatccg

SEQ ID NO: 48 - DNA, 39 bp, synthetic construct
  source 1..39: mol_type = other DNA
cgatgactcgtcgaaagacgaacagttnctttggatccg

SEQ ID NO: 49 - DNA, 39 bp, synthetic construct
  source 1..39: mol_type = other DNA
cgatgactcgtcgaaagacgaacagttcntttggatccg

SEQ ID NO: 50 - DNA, 39 bp, synthetic construct
  source 1..39: mol_type = other DNA
cgatgactcgtcgaaagacgaacagttccnttggatccg

SEQ ID NO: 51 - DNA, 39 bp, synthetic construct
  source 1..39: mol_type = other DNA
cgatgactcgtcgaaagacgaacagttcctntggatccg

SEQ ID NO: 52 - DNA, 38 bp, synthetic construct
  source 1..38: mol_type = other DNA
cgtctttcgacgagtcatcgcattcgtaacgctttcgc

SEQ ID NO: 53 - DNA, 38 bp, synthetic construct
  source 1..38: mol_type = other DNA
gatgactcgtcgaaagacgatcagttcctttggatccg

SEQ ID NO: 54 - DNA, 38 bp, synthetic construct
  source 1..38: mol_type = other DNA
tcgtctttcgacgagtcatcccattcgtaacgctttcg

SEQ ID NO: 55 - DNA, 38 bp, synthetic construct
  source 1..38: mol_type = other DNA
gatgactcgtcgaaagacgaacggttcctttggatccg

SEQ ID NO: 56 - DNA, 40 bp, synthetic construct
  source 1..40: mol_type = other DNA
tcgtctttcgacgagtcatcccattcgtaacgctttcgcg

SEQ ID NO: 57 - DNA, 41 bp, synthetic construct
  source 1..41: mol_type = other DNA
tgactcgtcgaaagacgaacagctcctttggatccgaattc

SEQ ID NO: 58 - DNA, 40 bp, synthetic construct
  source 1..40: mol_type = other DNA
gttcgtctttcgacgagtcatcccattcgtaacgctttcg

SEQ ID NO: 59 - DNA, 39 bp, synthetic construct
  source 1..39: mol_type = other DNA
ctcgtcgaaagacgaacagttgctttggatccgaattcg

SEQ ID NO: 60 - DNA, 38 bp, synthetic construct
  source 1..38: mol_type = other DNA
actgttcgtctttcgacgagtcatcccattcgtaacgc

SEQ ID NO: 61 - DNA, 38 bp, synthetic construct
  source 1..38: mol_type = other DNA
gatgactcgtcgaaagacgatcggttcctttggatccg

SEQ ID NO: 62 - DNA, 39 bp, synthetic construct
  source 1..39: mol_type = other DNA
tcgtctttcgacgagtcatcgcattcgtaacgctttcgc

SEQ ID NO: 63 - DNA, 38 bp, synthetic construct
  source 1..38: mol_type = other DNA
gatgactcgtcgaaagacgatcagctcctttggatccg

SEQ ID NO: 64 - DNA, 38 bp, synthetic construct
  source 1..38: mol_type = other DNA
gatgactcgtcgaaagacgatcagttgctttggatccg

SEQ ID NO: 65 - DNA, 38 bp, synthetic construct
  source 1..38: mol_type = other DNA
gatgactcgtcgaaagacgaacggctcctttggatccg

SEQ ID NO: 66 - DNA, 38 bp, synthetic construct
  source 1..38: mol_type = other DNA
gatgactcgtcgaaagacgaacggttgctttggatccg

SEQ ID NO: 67 - DNA, 38 bp, synthetic construct
  source 1..38: mol_type = other DNA
gatgactcgtcgaaagacgaacagctgctttggatccg

SEQ ID NO: 68 - DNA, 36 bp, synthetic construct
  source 1..36: mol_type = other DNA
tgactcgtcgaaagacgatcggttcctttggatccg

SEQ ID NO: 69 - DNA, 39 bp, synthetic construct
  source 1..39: mol_type = other DNA
gatcgtctttcgacgagtcatcccattcgtaacgctttc

SEQ ID NO: 70 - DNA, 37 bp, synthetic construct
  source 1..37: mol_type = other DNA
atgactcgtcgaaagacgatcagctcctttggatccg

SEQ ID NO: 71 - DNA, 39 bp, synthetic construct
  source 1..39: mol_type = other DNA
atcgtctttcgacgagtcatcccattcgtaacgctttcg

SEQ ID NO: 72 - DNA, 37 bp, synthetic construct
  source 1..37: mol_type = other DNA
atgactcgtcgaaagacgatcagttgctttggatccg

SEQ ID NO: 73 - DNA, 41 bp, synthetic construct
  source 1..41: mol_type = other DNA
atcgtctttcgacgagtcatcccattcgtaacgctttcgcg

SEQ ID NO: 74 - DNA, 38 bp, synthetic construct
  source 1..38: mol_type = other DNA
gtcgaaagacgaacggctcctttggatccgaattcgcc

SEQ ID NO: 75 - DNA, 39 bp, synthetic construct
  source 1..39: mol_type = other DNA
ggagccgttcgtctttcgacgagtcatcccattcgtaac

SEQ ID NO: 76 - DNA, 38 bp, synthetic construct
  source 1..38: mol_type = other DNA
cgaaagacgaacggttgctttggatccgaattcgccgc

SEQ ID NO: 77 - DNA, 41 bp, synthetic construct
  source 1..41: mol_type = other DNA
aagcaaccgttcgtctttcgacgagtcatcccattcgtaac

SEQ ID NO: 78 - DNA, 40 bp, synthetic construct
  source 1..40: mol_type = other DNA
gactcgtcgaaagacgaacagctgctttggatccgaattc

SEQ ID NO: 79 - DNA, 40 bp, synthetic construct
  source 1..40: mol_type = other DNA
tgttcgtctttcgacgagtcatcccattcgtaacgctttc

SEQ ID NO: 80 - DNA, 38 bp, synthetic construct
  source 1..38: mol_type = other DNA
ctcgtcgaaagacgatcggctcctttggatccgaattc

SEQ ID NO: 81 - DNA, 37 bp, synthetic construct
  source 1..37: mol_type = other DNA
gccgatcgtctttcgacgagtcatcgcattcgtaacg

SEQ ID NO: 82 - DNA, 37 bp, synthetic construct
  source 1..37: mol_type = other DNA
atgactcgtcgaaagacgatcggttgctttggatccg

SEQ ID NO: 83 - DNA, 38 bp, synthetic construct
  source 1..38: mol_type = other DNA
atcgtctttcgacgagtcatcgcattcgtaacgctttc

SEQ ID NO: 84 - DNA, 40 bp, synthetic construct
  source 1..40: mol_type = other DNA
cgtcgaaagacgaacggctgctttggatccgaattcgccg

SEQ ID NO: 85 - DNA, 40 bp, synthetic construct
  source 1..40: mol_type = other DNA
cagccgttcgtctttcgacgagtcatcgcattcgtaacgc

SEQ ID NO: 86 - DNA, 36 bp, synthetic construct
  source 1..36: mol_type = other DNA
tgactcgtcgaaagacgatcggctcctttggatccg

SEQ ID NO: 87 - DNA, 40 bp, synthetic construct
  source 1..40: mol_type = other DNA
gatcgtctttcgacgagtcatcccattcgtaacgctttcg

SEQ ID NO: 88 - DNA, 40 bp, synthetic construct
  source 1..40: mol_type = other DNA
ctcgtcgaaagacgatcggttgctttggatccgaattcgc

SEQ ID NO: 89 - DNA, 38 bp, synthetic construct
  source 1..38: mol_type = other DNA
accgatcgtctttcgacgagtcatcccattcgtaacgc

SEQ ID NO: 90 - DNA, 39 bp, synthetic construct
  source 1..39: mol_type = other DNA
tgggatgactcgtcgaaagacgatcagctgctttggatc

SEQ ID NO: 91 - DNA, 37 bp, synthetic construct
  source 1..37: mol_type = other DNA
tctttcgacgagtcatcccattcgtaacgctttcgcg

SEQ ID NO: 92 - DNA, 39 bp, synthetic construct
  source 1..39: mol_type = other DNA
tcgtcgaaagacgatcggctgctttggatccgaattcgc

SEQ ID NO: 93 - DNA, 38 bp, synthetic construct
  source 1..38: mol_type = other DNA
agccgatcgtctttcgacgagtcatcgcattcgtaacg

SEQ ID NO: 94 - DNA, 42 bp, synthetic construct
  source 1..42: mol_type = other DNA
tgactcgtcgaaagacgaacggctgctttggatccgaattcg

SEQ ID NO: 95 - DNA, 39 bp, synthetic construct
  source 1..39: mol_type = other DNA
gttcgtctttcgacgagtcatcccattcgtaacgctttc

SEQ ID NO: 96 - DNA, 40 bp, synthetic construct
  source 1..40: mol_type = other DNA
gactcgtcgaaagacgatcggctgctttggatccgaattc

SEQ ID NO: 97 - DNA, 40 bp, synthetic construct
  source 1..40: mol_type = other DNA
cgatcgtctttcgacgagtcatcccattcgtaacgctttc